SEQUENCE LISTING (1 sequence)

SEQ ID NO: 1 - DNA, 1436 bp, Lactobacillus acidophilus
  source 1..1436: mol_type = genomic DNA
tgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaa

(19) United States
(12) Patent Application Publication
(10) Pub. No.: US 2025/0043238 A1
(43) Pub. Date: Feb. 6, 2025

(54) LACTOBACILLUS ACIDOPHILUS GOLDGUT-LA100 HAVING FUNCTIONS OF LIPID LOWERING, BLOOD GLUCOSE LOWERING AND WEIGHT LOSS AND APPLICATION THEREOF

(71) Applicant: Shenzhen Porshealth Bioengineering Co., Ltd., Shenzhen (CN)

(72) Inventors: Yanyi Zheng, Shenzhen (CN); Silu Zhang, Shenzhen (CN); Guoxun Xiao, Shenzhen (CN); Tengxun Zhang, Shenzhen (CN); Yuebiao Feng, Shenzhen (CN); Xin Teng, Shenzhen (CN); Song Huang, Shenzhen (CN)

(21) Appl. No.: 18/919,965

(22) Filed: Oct. 18, 2024

(30) Foreign Application Priority Data
Oct. 20, 2023 (CN) 202311375894.8

(51) Int. Cl. C12N 1/20; A61K 35/747; C12R 1/23

(52) U.S. Cl. CPC C12N 1/205; A61K 35/747; C12R 2001/23

(57) ABSTRACT

The present invention relates to the technical field of microorganisms, in particular to a Lactobacillus acidophilus GOLDGUT-LA100 having functions of lipid lowering, blood glucose lowering and weight loss and application thereof. The Lactobacillus acidophilus GOLDGUT-LA100 provided by the present invention is preserved in China General Microbiological Culture Collection Center, with the preservation number of CGMCC No. 28398. The strain has strong acid resistance, can adapt to the digestive tract environment, and has high bile salt hydrolase activity. At the same time, in animal experiments, the strain significantly reduces blood glucose and serum cholesterol levels, reduces liver fat deposition, reduces adipose tissue inflammation, effectively reduces the weight, and has broad application prospects in the development of products for improving obesity, lowering lipid and lowering blood glucose.

### SEQUENCE LISTING

The sequence listing is submitted as a XML file filed via EFS-Web, with a file name of “Sequence_Listing.XML”, a creation date of Oct. 18, 2024, and a size of 3,650 bytes. The sequence Listing filed via EFS-Web is a part of the specification and is incorporated in its entirety by reference herein.

### TECHNICAL FIELD

The present invention relates to the technical field of microorganisms, in particular to a Lactobacillus acidophilus GOLDGUT-LA100 having functions of lipid lowering, blood glucose lowering and weight loss and application thereof.

### BACKGROUND

In recent years, the incidence of metabolic syndrome in humans has been increased globally, which increases the risk of coronary heart disease, stroke and other serious health problems. Obesity is a major factor that causes metabolic syndrome. Metabolic problems caused by obesity include hypertension, insulin resistance, type 2 diabetes, dyslipidemia and non-alcoholic fatty liver disease. With the improvement of the living standard of people and the change of lifestyles, the incidence of obesity has risen rapidly in the world, and has become a widely concerned health problem.

The main reason of obesity is the excess of energy intake over energy expenditure, and particularly related to the excessive intake of fats and refined carbohydrates. Before absorbed by the intestine, dietary fat is broken down into fatty acid, cholesterol and other products by the action of various enzymes and bile acids, and absorbed to enter the blood through the wall of the small intestine for the resynthesis of fat. In addition, when too much carbohydrate or sugar is taken in the diet, the excess sugar can also be converted into fat through a tricarboxylic acid cycle system. Excessive intake of the fat can cause an increase in serum cholesterol levels, and high cholesterol levels are an important factor that causes many cardiovascular diseases. Infiltrating macrophages in adipose tissue are associated with obesity-induced inflammation. The number of M1 macrophages is increased, proinflammatory cytokines are secreted, and inflammation is induced. Therefore, it is of great significance to reduce serum cholesterol levels and body fat percentage to prevent cardiovascular diseases.

The bile salt hydrolase (BSH) produced by intestinal bacteria can hydrolyze combined glycine bile salts and taurine bile salts into free bile salts and amino acid residues, thereby increasing the content of free bile salts in the intestinal cavity. Compared with the combined bile salts, the free bile salts have lower solubility and absorption efficiency. Therefore, the action of the BSH enables the reabsorption and the reuse of the bile salts, thereby increasing the synthesis of the bile salts in the liver. The synthesis of the bile salts in the liver requires the migration of cholesterol from the blood to the liver, thereby reducing the serum total cholesterol level. In addition, the lipid emulsifying capacity of the small intestine is limited, so the role of the BSH increases the excretion of dietary fat and cholesterol with feces. The BSH gene is widely present in lactic acid bacteria, such as Lactobacillus and Bifidobacterium, and has a deep exploration value in lowering cholesterol. Therefore, it is very necessary to screen lactic acid bacteria with high bile salt hydrolase activity.

### SUMMARY

The present invention provides a Lactobacillus acidophilus GOLDGUT-LA100 having functions of lipid lowering, blood glucose lowering and weight loss and application thereof.

The present invention conducts isolation and screening from the intestinal tract of healthy human bodies to obtain a strain of Lactobacillus acidophilus having high bile salt hydrolase activity and the functions of strong cholesterol lowering, weight loss and blood glucose lowering. The strain has the potential to further develop and prepare probiotic products such as functional food having the effects of lipid lowering, blood glucose lowering and weight loss.

Specifically, the present invention provides the following technical solution:

The present invention provides a Lactobacillus acidophilus GOLDGUT-LA100. The strain was preserved in China General Microbiological Culture Collection Center (CGMCC for short, at Institute of Microbiology, Chinese Academy of Sciences, No. 3, Yard 1, Beichen West Road, Chaoyang District, Beijing, postcode: 100101) on Sep. 8, 2023, with the classified name of Lactobacillus acidophilus and the preservation number of CGMCC No. 28398.

The Lactobacillus acidophilus GOLDGUT-LA100 is isolated from the intestinal tract of a healthy human body, and is identified by bacterial morphology, physiology and 16S rDNA sequencing. The result is Lactobacillus acidophilus, named Lactobacillus acidophilus GOLDGUT-LA100. The strain has good gastric acid tolerance and high bile salt hydrolase activity. In animal experiments, the strain can effectively reduce the weight, blood glucose and serum cholesterol levels of mice, reduce liver fat deposition, and play a role of resisting adipose tissue inflammation by affecting the differentiation of macrophages, so as to achieve the effects of relieving inflammation and losing weight to provide bacteria resources for the development of dietary supplements, health care products and other food and drugs with weight loss effect.

The Lactobacillus acidophilus GOLDGUT-LA100 has the following microbiological characteristics:

### (1) Morphological Features

Gram stain is positive, single or in pairs; colonies are formed on an MRS solid medium, which were milky white, smooth, convex, intact in edges, glittering and soft in texture.

### (2) Physiological Characteristics

The Lactobacillus acidophilus GOLDGUT-LA100 can grow well under artificial gastric acid conditions, and has high bile salt hydrolase activity. In animal experiments, the Lactobacillus acidophilus GOLDGUT-LA100 can effectively reduce the body weight, blood glucose and serum cholesterol levels of mice, reduce the liver fat deposition, and alleviate adipose tissue inflammation.

The Lactobacillus acidophilus GOLDGUT-LA100 can be cultured by the following method: inoculating the strain in the medium and culturing at 35-37° C. for 12-24 h. The medium can adopt the conventional medium in the field, which can enable the Lactobacillus acidophilus GOLDGUT-LA100 to grow therein, and preferably adopts an MRS broth medium. The composition of the MRS broth medium is as follows: 10.0 g/L of casease digest, 10.0 g/L of beef powder, 4.0 g/L of yeast powder, 2.0 g/L of triammonium citrate, 5.0 g/L of sodium acetate, 0.2 g/L of magnesium sulfate, 0.05 g/L of manganese sulfate, 20.0 g/L of glucose, 2.0 g/L of dipotassium hydrogen phosphate and 1.0 g/L of Twain 80, with pH=5.7±0.2, and 1.5% agar is added to the solid medium.

After safety evaluation, virulence gene analysis of the strain shows that the strain does not contain virulence factors. The results of antibiotic resistance tests show that the strain does not contain transferable antibiotic resistance genes. The results of animal toxicity experiments show that the behavior and mental condition of mice are good. The strain belongs to an actual non-toxic level.

The present invention provides a microbial preparation comprising the Lactobacillus acidophilus GOLDGUT-LA100.

Preferably, in the microbial preparation, the Lactobacillus acidophilus GOLDGUT-LA100 is present in the form of live bacteria.

The above microbial preparation may be a solid preparation (such as bacterial powder) or a liquid preparation (such as emulsion preparation).

The present invention provides a preparation method of the above microbial preparation, comprising a step for culturing the Lactobacillus acidophilus GOLDGUT-LA100.

Preferably, the culture is conducted at 35-37° C.

Preferably, the culture is conducted in a liquid medium to obtain a bacteria solution.

The above bacteria solution may be prepared into a liquid preparation directly or by concentrating and/or adding other excipients, or prepared into a solid preparation by drying, or prepared into a solid preparation by separating bacterial bodies in the bacteria solution and then preparing the bacterial bodies.

Based on the function of the Lactobacillus acidophilus GOLDGUT-LA100, the present invention provides the following application of the strain:

The present invention provides an application of the Lactobacillus acidophilus GOLDGUT-LA100 or the microbial preparation in preparation of a product for producing bile salt hydrolases.

The present invention provides an application of the Lactobacillus acidophilus GOLDGUT-LA100 or the microbial preparation in preparation of a product for increasing the content of bile salt hydrolases in a body.

The present invention provides an application of the Lactobacillus acidophilus GOLDGUT-LA100 or the microbial preparation in production of bile salt hydrolases.

The present invention provides an application of the Lactobacillus acidophilus GOLDGUT-LA100 or the microbial preparation in preparation of a product that has the functions of lowering lipid, reducing liver fat deposition, and/or relieving adipose tissue inflammation.

Preferably, the lipid lowering is the lowering of cholesterol, preferably the lowering of serum total cholesterol.

Preferably, the relief of adipose tissue inflammation is achieved by improving the infiltration of macrophages in the fat.

The present invention provides an application of the Lactobacillus acidophilus GOLDGUT-LA100 or the microbial preparation in preparation of a product for lowering blood glucose.

The present invention provides an application of the Lactobacillus acidophilus GOLDGUT-LA100 or the microbial preparation in preparation of a product for weight loss.

Preferably, the weight loss is to inhibit weight gain and/or reduce weight.

Preferably, the action objects of the product for weight loss are high-fat diet animals, which may be healthy or obese animals.

In the above application, the product is food, a drug, a feed or a feed additive.

The present invention provides an application of the Lactobacillus acidophilus GOLDGUT-LA100 or the microbial preparation in preparation of food, a drug, a feed or a feed additive.

In the present invention, the food comprises ordinary food, dietary supplements and health products.

The present invention provides a product comprising the Lactobacillus acidophilus GOLDGUT-LA100 or the microbial preparation; and the product is food, a drug, a feed or a feed additive.

In addition to comprising the Lactobacillus acidophilus GOLDGUT-LA100 or the microbial preparation, the food may also comprise raw materials permitted in the field of food. The food may be dietary supplements, health products or fermented food.

In addition to comprising the Lactobacillus acidophilus GOLDGUT-LA100 or the microbial preparation, the drug may also comprise ingredients (e.g., fillers, excipients, lubricants, wetting agents, diluents, etc.) permitted in the field of pharmacy. The preparation type of the drug may be a solid preparation (e.g. powder, granule, capsule, tablet, etc.) or a liquid preparation (e.g. oral liquid, etc.).

The beneficial effects of the present invention include at least: the Lactobacillus acidophilus GOLDGUT-LA100 provided by the present invention has strong acid resistance, can adapt to the digestive tract environment, and has high bile salt hydrolase activity. At the same time, in animal experiments, the Lactobacillus acidophilus GOLDGUT-LA100 significantly reduces blood glucose and serum cholesterol levels, reduces liver fat deposition, reduces adipose tissue inflammation, effectively reduces the weight, and has broad application prospects in the development of products for reducing weight or improving obesity, lowering lipid and lowering blood glucose.

### DESCRIPTION OF DRAWINGS

To more clearly describe the technical solutions in the present invention or in the prior art, the drawings required to be used in the description of the embodiments or the prior art will be simply presented below. Obviously, the drawings in the following description are some embodiments of the present invention, and for those ordinary skilled in the art, other drawings can also be obtained according to these drawings without contributing creative labor.

FIG. 1 shows the colony morphology of Lactobacillus acidophilus GOLDGUT-LA100 in embodiment 1 of the present invention.

FIG. 2 shows the gram staining diagram of Lactobacillus acidophilus GOLDGUT-LA100 in embodiment 1 of the present invention with a magnification of 1000 times.

FIG. 3 shows the survival rate of Lactobacillus acidophilus GOLDGUT-LA100 and control strains in embodiment 2 of the present invention after cultured in artificial gastric acid for 2 h; the data are from three repeated experiments, and the error line represents the standard deviation.

FIG. 4 shows the yields of glycine (A) and taurine (B) catalyzed by bile salt hydrolase of Lactobacillus acidophilus GOLDGUT-LA100 and control strains in embodiment 3 of the present invention; data are from three repeated experiments, and the error line represents the standard deviation; the statistical analysis method is Student's t-test; ** represents that p value is less than 0.01, and **** represents that p value is less than 0.0001.

FIG. 5 shows the weight change of mice in each group in embodiment 4 of the present invention, wherein A shows the weight change of mice in four groups from week 1 to week 14; B shows the weight values of mice in four groups at week 14; C shows the weight gain rate of mice in four groups from week 8 to week 14; the error line represents the standard deviation; the statistical analysis method in B and C is one-way analysis of variance; * represents that p value is less than 0.05, ** represents that p value is less than 0.01, and **** represents that p value is less than 0.0001.

FIG. 6 shows the blood glucose values of mice in four groups after intervention in embodiment 5 of the present invention; the statistical analysis method is one-way analysis of variance; * represents that p value is less than 0.05 and *** represents that p value is less than 0.001.

FIG. 7 shows the serum total cholesterol content of mice in four groups after intervention in embodiment 6 of the present invention; the statistical analysis method is one-way analysis of variance; * represents that p value is less than 0.05 and **** represents that p value is less than 0.0001.

FIG. 8 shows the oil red O fat staining diagram of livers of mice in four groups after intervention in embodiment 7 of the present invention.

FIG. 9 shows the flow cytometry detection result diagram (A) and M1 macrophage quantitative result diagram (B) in embodiment 8 of the present invention; the statistical analysis method is one-way analysis of variance; ** represents that p value is less than 0.01, **** represents that p value is less than 0.0001, and ns represents that there is no significant difference.

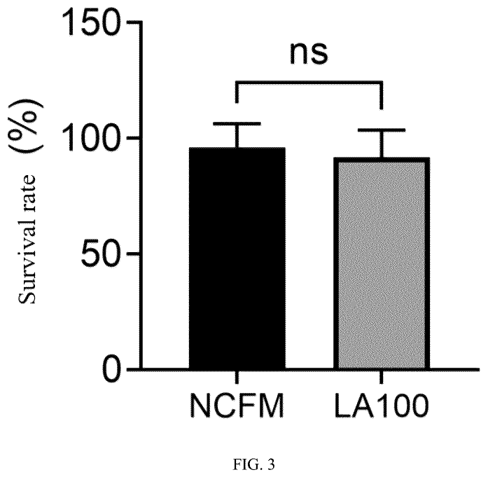

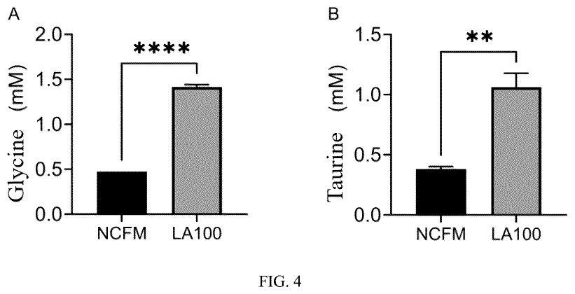

In FIGS. 3 and 4, NCFM represents the control strain, and LA100 represents Lactobacillus acidophilus GOLDGUT-LA100.

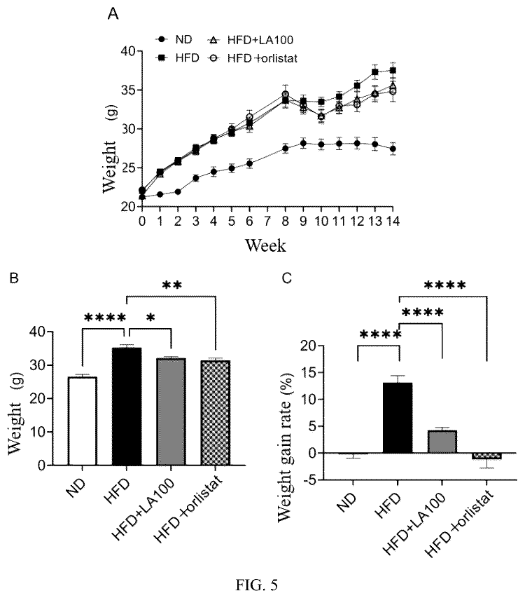

In FIGS. 5, 6, 7, 8 and 9, ND represents a normal diet group, HFD represents a high-fat diet group, HFD+LA100 represents a Lactobacillus acidophilus GOLDGUT-LA100 intervention group, and HFD+orlistat represents a positive drug intervention group.

### DETAILED DESCRIPTION

To make purposes, technical solutions and advantages of the present invention more clear, the technical solutions in the present invention will be clearly and fully described below in combination with the drawings in the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1 Isolation and Identification of Lactobacillus acidophilus GOLDGUT-LA100

1. Isolation of Lactobacillus acidophilus GOLDGUT-LA100
1.1 Sample Source

The Lactobacillus acidophilus of the present invention is isolated from a healthy human gut.

### 1.2 Preparation of Medium

The medium used for sample isolation and strain screening is an MRS solid medium. The components of an MRS liquid medium are as follows: 10.0 g/L of casease digest, 10.0 g/L of beef powder, 4.0 g/L of yeast powder, 2.0 g/L of triammonium citrate, 5.0 g/L of sodium acetate, 0.2 g/L of magnesium sulfate (MgSO₄·7H₂O), 0.05 g/L of manganese sulfate (MnSO₄·4H₂O), 20.0 g/L of glucose, 2.0 g/L of dipotassium hydrogen phosphate, and 1.0 g/L of Twain −80, and 1.5% agar is added to prepare the MRS solid medium, with pH=5.7±0.2.

### 1.3 Isolation of Strains

1 g of sample was put into 10 mL of MRS liquid medium prepared in step 1.2, mixed evenly and then cultured at 36° C.; then 1 mL of enrichment solution is absorbed in a super clean bench for 10-fold gradient dilution; 100 μL of bacteria solution with 10⁻⁵, 10⁻⁶ and 10⁻⁷ dilution gradients was selected and coated on a petri dish containing the sterile MRS solid medium, and subjected to stationary culture at 36° C. under aerobic conditions for 24-48 h; and after obvious single colonies were formed, typical colonies were selected, and scribed and purified on the MRS solid plate medium for several times until the cultures with consistent colony morphology on the whole plate were identified for strains.

2. Identification of Lactobacillus acidophilus GOLDGUT-LA100

### 2.1 Colony Features

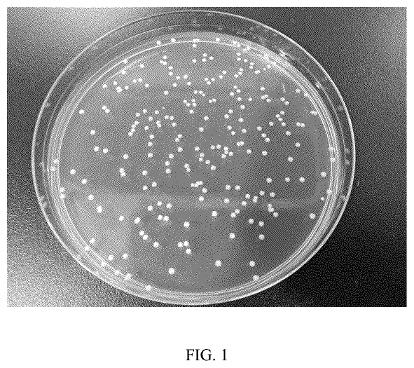

After the Lactobacillus acidophilus GOLDGUT-LA100 was cultured in the MRS solid medium for 24 h, the colonies were small, milky white, smooth, convex, intact in edges, glittering and soft in texture, as shown in FIG. 1.

### 2.2 Microscopic Morphology

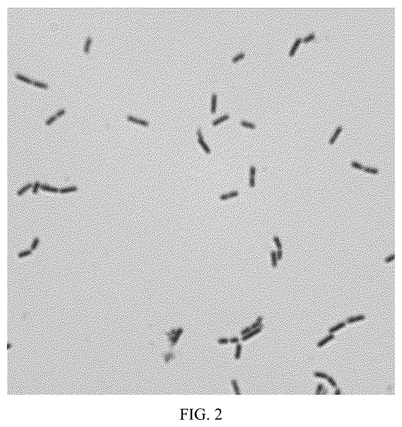

The gram stain for Lactobacillus acidophilus GOLDGUT-LA100 is positive, single or paired, and V-shaped, as shown in FIG. 2.

2.3 16S rDNA Identification

Identification sequence: as shown in SEQ ID NO.1.

Identification results: According to the combination of the 16S rDNA sequence comparison results and physiological and biochemical results of the strain, the strain was determined to be Lactobacillus acidophilus.

Lactobacillus acidophilus GOLDGUT-LA100 was preserved in China General Microbiological Culture Collection Center (CGMCC for short, at Institute of Microbiology, Chinese Academy of Sciences, No. 3, Yard 1, Beichen West Road, Chaoyang District, Beijing, postcode: 100101) on Sep. 8, 2023, with the classified name of Lactobacillus acidophilus and the preservation number of CGMCC No. 28398.

### Embodiment 2 Detection of Gastric Acid Tolerance of Lactobacillus acidophilus GOLDGUT-LA100

The overall pH condition of the human stomach environment is strongly acidic, so the acid resistance of the strain is an important index to evaluate whether the strain can survive and colonize in the stomach acid environment. Artificial gastric juice was used to simulate the gastric environment. 100 μL of Lactobacillus acidophilus GOLDGUT-LA100 bacteria solution and 100 μL of simulated artificial gastric juice were blown and mixed evenly, and co-incubated anaerobically for 2 h at 37° C. Samples were taken at 0 h and 2 h respectively. The bacteria solution was diluted by 10-fold continuous gradient and coated on the MRS solid medium. After anaerobic culture at 37° C. for 48 h, colony count was performed, and the survival rate of the bacteria was calculated by a calculation formula: survival rate (%)=(2 h viable count/0 h viable count)×100%. The control strain was Lactobacillus acidophilus NCFM, which was a widely used edible probiotic strain with excellent acid resistance. It can be seen from FIG. 3 that Lactobacillus acidophilus GOLDGUT-LA100 has strong stomach acid tolerance, and the survival rate after treatment with artificial gastric juice is not significantly different from that of Lactobacillus acidophilus NCFM.

### Embodiment 3 Determination of Bile Salt Hydrolase Activity of Lactobacillus acidophilus GOLDGUT-LA100

The present embodiment characterizes the activity of bile salt hydrolase by measuring the amount of amino acids (taurine and glycine) produced by catalyzing bile salt hydrolysis by bile salt hydrolase.

Lactobacillus acidophilus GOLDGUT-LA100 bacteria solution and the Lactobacillus acidophilus NCFM bacteria solution of the control strain were inoculated into 10 mL of MRS liquid medium with the inoculation ratio of 5% (v/v), and subjected to stationary anaerobic overnight culture at 37° C. for more than 16 h. The bacteria solution was centrifuged at 6000 rpm at 4° C. for 10 min; the supernatant was discarded; the bacteria were resuspended and washed by oscillating with the same volume of PBS, and centrifuged at 6000 rpm for 10 min. The washing operation was repeated twice, and the bacteria obtained by the last centrifugation was resuspended with 3 mL of PBS. After resuspension of the bacteria solution, the OD value of the bacteria solution was measured with a disposable colorimetric dish, and the same OD value was adjusted with ultrapure water. 3 mL of Lactobacillus acidophilus GOLDGUT-LA100 bacteria solution and the Lactobacillus acidophilus NCFM bacteria solution of the control strain with the same OD were taken into 10 mL centrifugal tubes, and 4 glass beads were added into each tube. Then, the centrifugal tubes were put into a tissue grinder to work at 70 Hz for 60 times (working for 20 s and stopping for 10 s). The ground sample was swirled, oscillated and mixed evenly. 1 mL was taken in a 2 mL centrifugal tube, and centrifuged at 12000 rpm at 4° C. for 5 min. The supernatant was collected. 950 μL of supernatant was taken into a 2 mL centrifugal tube; 50 μL of 0.4 M bile salt was added into the centrifugal tube; and the mixed system was heated in a water bath at 37° C. for 30 min. After the reaction, 500 μL of 15% trichloroacetic acid was added, swirled, oscillated, mixed evenly and centrifugated at 12000 rpm for 5 min. 200 μL of centrifugal supernatant was taken into a 10 mL centrifugal tube; 1 mL of ninhydrin reaction solution and 1 mL of 0.5M citric acid buffer (pH=5.5) were added into the centrifugal tube, swirled, oscillated, mixed evenly, and heated in a boiling water bath for 30 min until the color of the system was stable. The system after the boiling water bath was cooled in cold water, 2.8 mL of 70% ethanol was added to the system, and the system was swirled, oscillated and mixed evenly. The sample was transferred to a 96-well plate with 200 μL per well, and the sample OD₅₇₀ was measured by a microplate reader and the data were recorded. The absorbance values obtained were substituted into the standard curves of glycine and taurine to calculate the production of two amino acids. It can be seen from FIG. 4 that, compared with the control strain group, Lactobacillus acidophilus GOLDGUT-LA100 group produces more glycine and taurine, which proves that the bile salt hydrolase activity of Lactobacillus acidophilus GOLDGUT-LA100 was higher than that of the control strain.

### Embodiment 4 Weight Loss Effect of Lactobacillus acidophilus GOLDGUT-LA100 in a Mouse Model

C57BL/6 mice aged 4-6 weeks were randomly divided into a high-fat diet (HFD) group and a normal diet (ND) group. Each group was provided with different diet types. The diet of the HFD group was 20% of kcal protein, 20% of kcal carbohydrate and 60% of kcal fat, with 5.24 kcal/g. The diet of the ND group was 20.6% of kcal protein, 67.4% of kcal carbohydrate and 12% of kcal fat, with 3.64 kcal/g. Weight was measured weekly and each group was fed for 7 weeks. Obese modeling mice were selected from the HFD group according to the standard that the average weight of mice was higher by 20% than the ND group, and intervention experiments were carried out.

The obese modeling mice were divided into three groups: HFD+GOLDGUT-LA100 groups were given probiotic gavage intervention, that is, Lactobacillus acidophilus GOLDGUT-LA100 resuspended with normal saline (2×10E9 CFU/mL, 500 μL/day/mouse) was used for gavage by a mouse gavage needle. HFD+orlistat groups were intervened with positive drug orlistat by gavage, i.e., orlistat dissolved with normal saline was gavaged by a mouse gavage needle (80 mg/kg/day, 500 μL/mouse). The HFD group was used as control group. The same amount of normal saline was gavaged. The intervention lasted for 7 weeks, during which all three groups were given the same high-fat diet. The ND group is used as a blank control group, and the mice were gavaged with the same amount of normal saline for 7 weeks and provided with the same normal diet. The weight was measured weekly.

A in FIG. 5 shows the 14-week weight change of the mice in four groups. Compared with the HFD control group, both the probiotic GOLDGUT-LA100 and the positive drug intervention significantly reduce the weight of the mice at week 14 (B in FIG. 5). According to the weight gain rate during the intervention (weight gain rate=(weight at week 14−weight at week 8)*100%/weight at week 8), the intervention of Lactobacillus acidophilus GOLDGUT-LA100 also significantly reduces the weight gain rate (C in FIG. 5). The above results show that the intervention of strain GOLDGUT-LA100 can slow the weight gain of obese mice induced by high-fat diet.

### Embodiment 5 Effect of Lowering Blood Glucose by Lactobacillus acidophilus GOLDGUT-LA100 in a Mouse Model

The mice in embodiment 4 were taken. After the intervention at week 14, trace blood samples were taken by a tail blood extraction method and blood glucose levels were measured with a glucose meter.

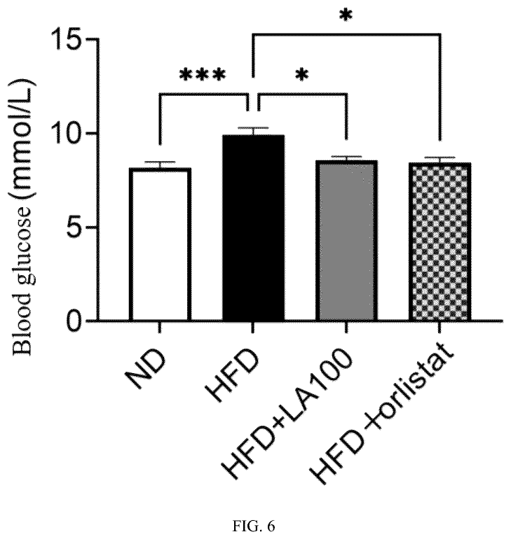

The results show that the intervention of the probiotic GOLDGUT-LA100 lowers the blood glucose levels in the mice compared with the mice in the HFD group (FIG. 6).

### Embodiment 6 Fat Reduction Effect of Lactobacillus acidophilus GOLDGUT-LA100 in a Mouse Model

The mice in embodiment 4 were taken. After the intervention at week 14, blood was collected from the eyeballs to isolate the serum, and the total cholesterol content in the blood was detected by an automatic biochemical analyzer.

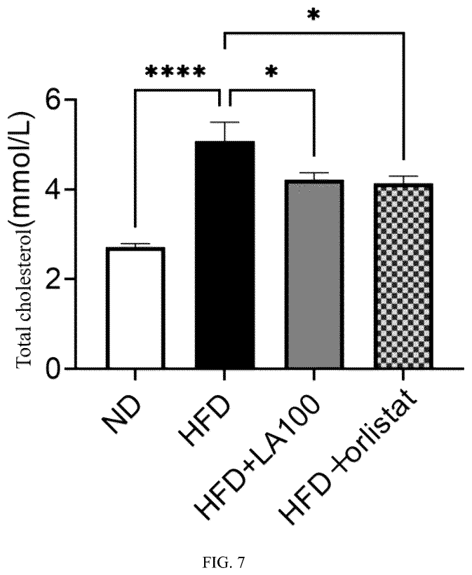

The results show that the intervention of the probiotic GOLDGUT-LA100 reduces the total blood cholesterol content of the mice compared with the mice in the HFD group (FIG. 7).

### Embodiment 7 Reduction of Liver Fat Deposition by Lactobacillus acidophilus GOLDGUT-LA100 in a Mouse Model

The mice in embodiment 4 were taken. After the intervention at week 14, the liver was dissected, and tissue sections were made by cherry blossom embedding, stained with oil red O, observed with a microscope and photographed.

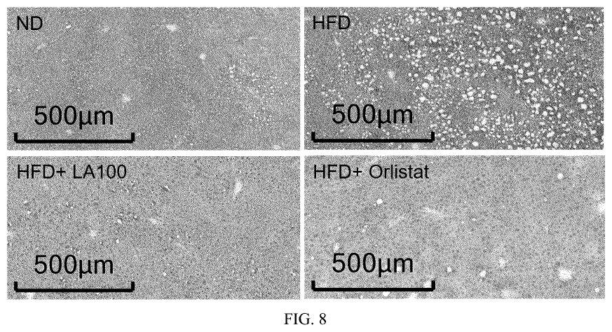

It can be seen from FIG. 8 that the high-fat diet causes the deposition of liver fat, and the intervention of the positive drug orlistat and probiotic GOLDGUT-LA100 has the effect of reducing liver fat deposition.

### Embodiment 8 Improvement of Macrophage Infiltration in Fat in a Mouse Model by Lactobacillus acidophilus GOLDGUT-LA100

The mice in embodiment 4 were taken. After the intervention at week 14, the adipose tissue was cut into 2 mm×2 mm chunks with ophthalmic scissors; type II collagenase was added; the mixture was put in a water bath at 37° C. for 30 min; after the adipose tissue was digested into chylous shape, the adipose tissue was centrifuged at 1500 rpm for 5 min; the upper adipose tissue was discarded; and the cells were washed by PBS and resuspended. The adipose tissue resuspending cells after antibody staining were detected by a flow cytometry. Antibodies were selected as follows: an allophycocyanin anti-mouse F4/80 antibody, a peridinin chlorophyll protein anti-mouse CD11c antibody, and a fluorescein isothiocyanate anti-mouse CD11b antibody. F4/80+CD11c+CD11b+ was defined as M1 macrophage; and F4/80+CD11c+CD11b− was defined as M2 macrophage. The infiltration of the macrophages in the adipose tissue was calculated by the flow cytometry.

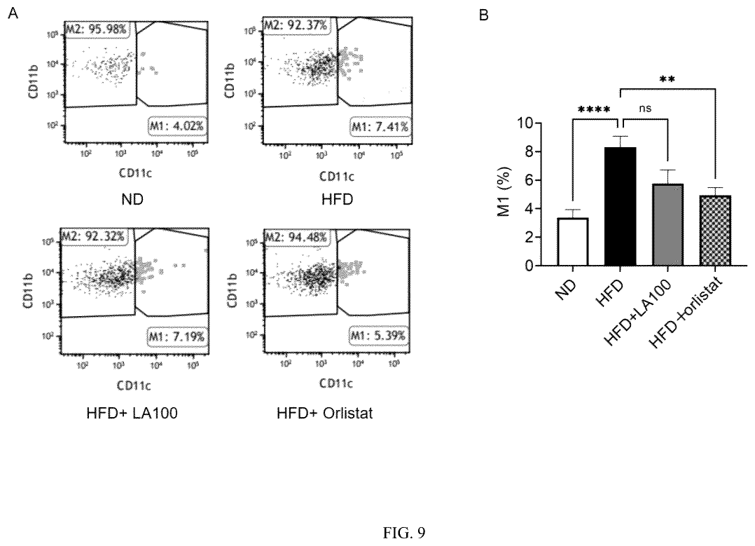

It can be seen from the flow cytometry detection results of A in FIG. 9 and the quantitative results of M1 macrophages in B in FIG. 9 that the positive drug orlistat significantly reduces the number of the M1 macrophages and reduces the inflammation of the adipose tissue. The intervention of the probiotic GOLDGUT-LA100 can also resist fatty inflammation induced by the high-fat diet.

In conclusion, the present invention obtains a strain of Lactobacillus acidophilus through isolation and screening, and the strain is named Lactobacillus acidophilus GOLDGUT-LA100. The strain can tolerate the artificial gastric acid environment and adapt to the digestive tract environment, and has high bile salt hydrolase activity. In vivo experiments in the mice demonstrate that the colonization of the strain can significantly reduce blood glucose and serum cholesterol levels, reduce liver fat deposition, reduce adipose tissue inflammation, and reduce the weight gain induced by the high-fat diet. Thus, the strain has broad application prospects in products for improving obesity, lowering lipid and lowering blood glucose.

Finally, it should be noted that the above embodiments are only used to illustrate the technical solutions of the present invention, not to limit the technical solutions. Although the present invention is described in detail by referring to the above embodiments, those ordinary skilled in the art shall understand that the technical solutions recorded in the above embodiments can be still amended, or some technical features therein can be replaced equivalently. These amendments or replacements do not enable the essence of the corresponding technical solutions to depart from the spirit and the scope of the technical solutions in the embodiments of the present invention.

What is claimed is:

1. A Lactobacillus acidophilus GOLDGUT-LA100, preserved in China General Microbiological Culture Collection Center, with the preservation number of CGMCC No. 28398.

2. A microbial preparation, comprising the Lactobacillus acidophilus GOLDGUT-LA100 according to claim 1.

3. A preparation method of the microbial preparation according to claim 2, comprising a step for culturing the Lactobacillus acidophilus GOLDGUT-LA100.

4. The method according to claim 3, wherein the culture is conducted at 35-37° C.

5. An application of the Lactobacillus acidophilus GOLDGUT-LA100 according to claim 1 in preparation of a product for producing bile salt hydrolases.

6. An application of the microbial preparation according to claim 2 in preparation of a product for producing bile salt hydrolases.

7. An application of the Lactobacillus acidophilus GOLDGUT-LA100 according to claim 1 in preparation of a product that has the function of lowering lipid.

8. An application of the microbial preparation according to claim 2 in preparation of a product that has the function of lowering lipid.

9. An application of the Lactobacillus acidophilus GOLDGUT-LA100 according to claim 1 in preparation of a product for lowering blood glucose.

10. An application of the microbial preparation according to claim 2 in preparation of a product for lowering blood glucose.

11. An application of the Lactobacillus acidophilus GOLDGUT-LA100 according to claim 1 in preparation of a product for weight loss.

12. An application of the microbial preparation according to claim 2 in preparation of a product for weight loss.

13. An application of the Lactobacillus acidophilus GOLDGUT-LA100 according to claim 1 in preparation of food, a drug, a feed or a feed additive.

14. An application of the microbial preparation according to claim 2 in preparation of food, a drug, a feed or a feed additive.

15. A product, comprising the Lactobacillus acidophilus GOLDGUT-LA100 according to claim 1;
wherein the product is food, a drug, a feed or a feed additive.

16. A product, comprising the microbial preparation according to claim 2;
wherein the product is food, a drug, a feed or a feed additive.
